(19) United States
(12) Patent Application Publication
(10) Pub. No.: US 2019/0030369 A1
(43) Pub. Date: Jan. 31, 2019

(54) THERAPY PLANNING SYSTEM FOR NEUTRON CAPTURE THERAPY

(71) Applicant: SUMITOMO HEAVY INDUSTRIES, LTD., Tokyo (JP)

(72) Inventor: Tetsuya Mukawa, Kanagawa (JP)

(21) Appl. No.: 16/138,355

(22) Filed: Sep. 21, 2018

Related U.S. Application Data
(63) Continuation of application No. PCT/JP2017/013353, filed on Mar. 30, 2017.

(30) Foreign Application Priority Data
Mar. 31, 2016 (JP) 2016-070411

(51) Int. Cl. A61N 5/10

(52) U.S. Cl. CPC A61N 5/1031; A61N 5/1064; A61N 5/1071; A61N 5/1045; A61N 2005/109

(57) ABSTRACT

Provided is a therapy planning system for neutron capture therapy performing therapy planning thereof irradiating an irradiation target with a neutron beam. The system includes an image acquiring unit acquiring an image of the irradiation target; a body outline setting unit setting a body outline of the irradiation target based on the image acquired by the image acquiring unit; a vacancy region setting unit setting a part where a value of a pixel of the image is within a range set in advance on an inner side of the body outline set by the body outline setting unit, as a vacancy region including a vacancy; and a mucosa region setting unit setting apart on an outer side by a first thickness from a surface of the vacancy region set by the vacancy region setting unit on the inner side of the body outline, as a mucosa region including mucosa.

### RELATED APPLICATIONS

Priority is claimed to Japanese Patent Application No. 2016-070411, filed Mar. 31, 2016, and International Patent Application No. PCT/JP2017/013353, the entire content of each of which is incorporated herein by reference.

### BACKGROUND

### Technical Field

A certain embodiment of the present invention relates to a therapy planning system for neutron capture therapy.

### Description of Related Art

In the related art, radiation therapy using radial rays has been performed. A therapy planning system, which performs planning for irradiating a lesion with radial rays in a stage prior to such radiation therapy, is known (for example, refer to the related art). Here, as a therapeutic method using radial rays, boron neutron capture therapy (BNCT), which is neutron capture therapy irradiating cancer cells with a neutron beam and annihilates the cancer cells, is known. In boron neutron capture therapy, boron that has been injected into cancer cells in advance is irradiated with a neutron beam so that the cancer cells are selectively destroyed due to dispersion of heavy charged particles generated through the irradiation.

### SUMMARY

According to an embodiment of the present invention, there is provided a therapy planning system for neutron capture therapy performing therapy planning of the neutron capture therapy in which an irradiation target is irradiated with a neutron beam. The therapy planning system for neutron capture therapy includes an image acquiring unit configured to acquire an image of the irradiation target; a body outline setting unit configured to set a body outline of the irradiation target based on the image acquired by the image acquiring unit; a vacancy region setting unit configured to set apart in which a value of a pixel of the image is within a range set in advance on an inner side of the body outline set by the body outline setting unit, as a vacancy region in which a vacancy is disposed; and a mucosa region setting unit configured to set a part on an outer side of the vacancy region by a first thickness from a surface of the vacancy region set by the vacancy region setting unit on the inner side of the body outline, as a mucosa region in which mucosa is disposed.

### BRIEF DESCRIPTION OF THE DRAWINGS

FIG. 1 is a view illustrating a therapy planning system for neutron capture therapy and a disposed neutron capture therapy apparatus of the present embodiment.

FIG. 2 is a view illustrating a part in the vicinity of a neutron beam irradiation unit of the neutron capture therapy apparatus in FIG. 1.

FIG. 3 is a block configuration diagram of the therapy planning system for neutron capture therapy in FIG. 1.

FIGS. 4A to 4C are views illustrating each of regions in an image.

FIGS. 5A and 5B are schematic views illustrating a method of setting a skin region.

FIGS. 6A and 6B are schematic views illustrating a method of setting a mucosa region.

FIG. 7 is a flowchart illustrating a procedure of therapy planning.

### DETAILED DESCRIPTION

In radiation therapy, there is a need to grasp the influence of irradiation of radial rays not only on a lesion but also on normal tissue. In addition, in neutron capture therapy, in order to grasp the influence on normal tissue, the atomic composition which varies every tissue needs to be taken into consideration. Therefore, a therapy planning system of radiation therapy in the related art cannot be used in neutron capture therapy in an intact manner, and there has been a demand for a therapy planning system suitable for neutron capture therapy.

It is desirable to provide a therapy planning system for neutron capture therapy capable of performing therapy planning suitable for neutron capture therapy.

Here, since neutron capture therapy is a therapeutic method utilizing nuclear reaction between a neutron beam and atomic nuclei of an irradiation object, a radiation dose distribution needs to be calculated using a model considering a region based on the atomic composition of an irradiation target. In addition, since a part of an irradiation target corresponding to mucosa is highly sensitive to radial rays, when therapy planning is performed, the therapy planning needs to be performed after setting a region in which mucosa is disposed.

Therefore, the therapy planning system for neutron capture therapy according to the embodiment of the present invention includes the vacancy region setting unit configured to set a part in which a value of a pixel of an image is within a predetermined range on the inner side of the body outline set by the body outline setting unit, as the vacancy region in which a vacancy is disposed. In an image acquired by the image acquiring unit, the part in which the value of the pixel is within the predetermined range is a part on an outer side of the body outline of the irradiation target, or a part in which a vacancy is disposed inside the irradiation target. Therefore, the vacancy region can be set by the body outline setting unit configured to set a body outline and the vacancy region setting unit configured to determine the value of the pixel on the inner side of the body outline. Here, mucosa is disposed near the surface of an inner wall part of the vacancy. Thus, the mucosa region setting unit can set a part on the outer side of the vacancy region by the first thickness from the surface of the vacancy region set by the vacancy region setting unit, as the mucosa region in which mucosa is disposed. In this manner, the mucosa region can be easily set by setting the vacancy region. Therapy planning can be performed based on the radiation dose of a neutron beam with respect to the mucosa region. Consequently, therapy planning suitable for neutron capture therapy can be performed.

The therapy planning system for neutron capture therapy may further include a bone region setting unit configured to set a part in which a bone is disposed on the inner side of the body outline, as a bone region; and an organ region setting unit configured to set a part in which an organ is disposed on the inner side of the body outline, as an organ region. The mucosa region setting unit may delete a part of the set mucosa region overlapping at least one of the bone region and the organ region from the mucosa region. Since bones and organs are formed of an atomic composition different from that of mucosa, the mucosa region is set as a region different from a bone region and an organ region. Therefore, the mucosa region can be more accurately set by deleting apart overlapping at least one of the bone region and the organ region from the mucosa region.

The therapy planning system for neutron capture therapy may further include a skin region setting unit configured to set a part on an inner side of the body outline by a second thickness from the body outline, as a skin region in which skin is disposed. Not only mucosa but also skin is a part in which the allowable radiation dose of an irradiation neutron beam is restricted. In addition, skin is disposed at a part on the inner side of the body outline by the second thickness from the body outline. In this manner, the skin region can be easily set by setting the body outline. Therapy planning can be performed based on the radiation dose of a neutron beam with respect to the skin region. Therefore, more suitable therapy planning can be performed by setting the skin region.

The therapy planning system for neutron capture therapy may further include an air region setting unit configured to set an entire region on the outer side of the body outline, as an air region. Accordingly, even in a case where a therapy table or the like is disposed, a load of computation can be reduced by the air region setting unit configured to set the entire region on the outer side of the body outline as the air region.

According to the present invention, therapy planning suitable for neutron capture therapy can be performed.

Hereinafter, with reference to the accompanying drawings, a therapy planning system for neutron capture therapy according to the present invention, and a neutron capture therapy apparatus including the therapy planning system will be described. In description of the drawings, the same reference signs will be applied to the same elements, and duplicated description will be omitted.

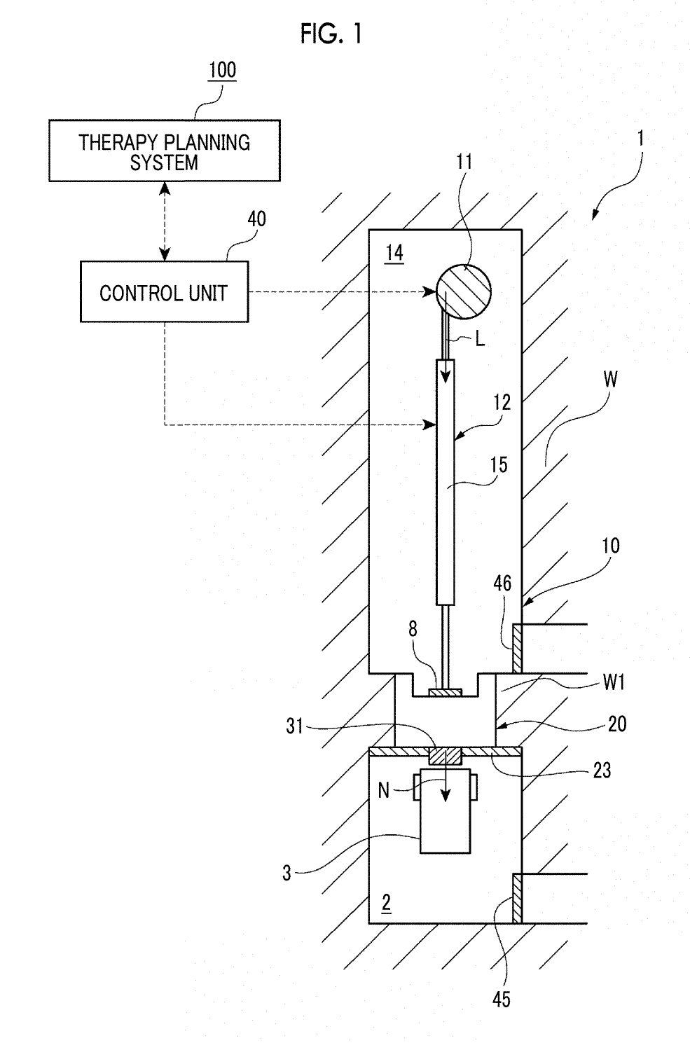

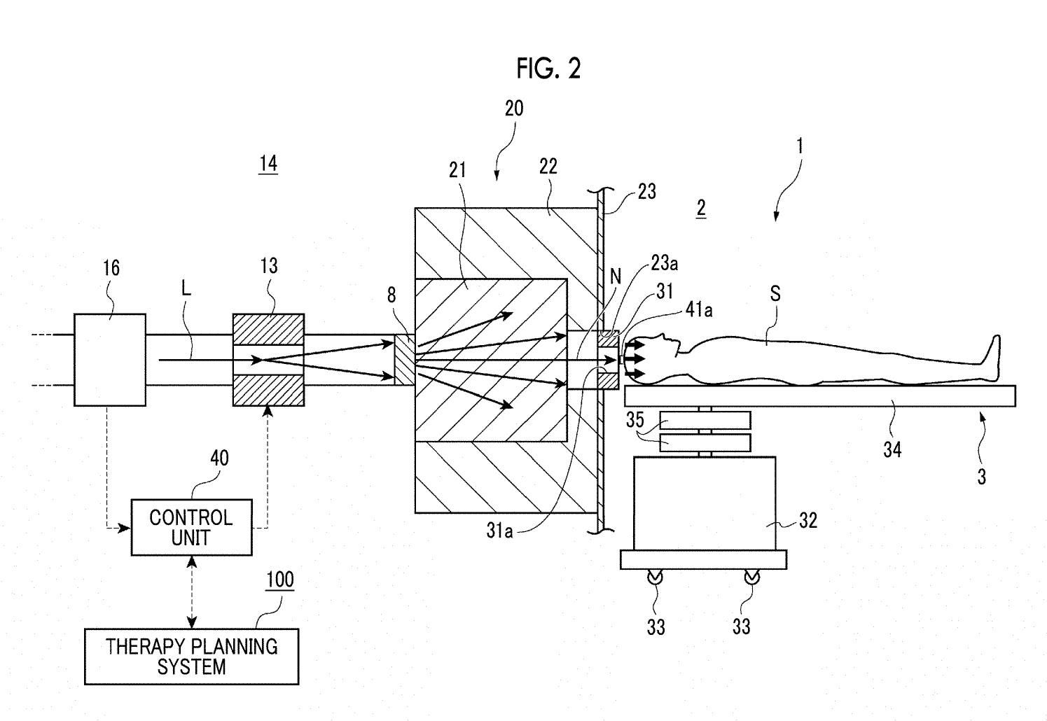

First, using FIGS. 1 and 2, an overview of the neutron capture therapy apparatus according to the present embodiment will be described. As illustrated in FIGS. 1 and 2, a neutron capture therapy apparatus 1 performing cancer therapy using boron neutron capture therapy is an apparatus which performs cancer therapy by irradiating a site of a patient (irradiation target) S, to which boron (10B) is applied and the boron is clustered, with a neutron beam. The neutron capture therapy apparatus 1 has an irradiation chamber 2 in which cancer therapy of the patient S is performed by irradiating the patient S bound to a therapy table 3 with a neutron beam N.

Preparation work in which the patient S is bound to the therapy table 3 is carried out in a preparation room (not illustrated) outside the irradiation chamber 2, and the therapy table 3, to which the patient S is bound, is moved from the preparation room to the irradiation chamber 2. In addition, the neutron capture therapy apparatus 1 includes a neutron beam generation unit 10 that generates the neutron beam N for therapy, and a neutron beam irradiation unit 20 that irradiates the patient S bound to the therapy table 3 inside the irradiation chamber 2 with the neutron beam N. The irradiation chamber 2 is covered with a blockade wall W. A passage and a door 45 through which a patient, a worker, or the like passes may be provided.

The neutron beam generation unit 10 includes an acceleration unit 11 which accelerates charged particles and causes a charged particle beam L to exit, a beam transportation path 12 through which the charged particle beam L caused to exit by the acceleration unit 11 is transported, a charged particle beam scanning unit 13 which controls the irradiation position of the charged particle beam L with respect to a target 8 by performing scanning with the charged particle beam L, the target 8 which causes nuclear reaction to irradiation of the charged particle beam L and generates the neutron beam N, and a current monitor 16 which measures a current of the charged particle beam L. The acceleration unit 11 and the beam transportation path 12 are disposed inside a charged particle beam generation chamber 14 having a substantially rectangular shape, and this charged particle beam generation chamber 14 is a space covered with the blockade wall W made of concrete. A passage and a door 46 through which a worker passes for maintenance may be provided in the charged particle beam generation chamber 14. The shape of the charged particle beam generation chamber 14 is not limited to a substantially rectangular shape. The charged particle beam generation chamber 14 may have a different shape. For example, in a case where a route from the acceleration unit to a target has an L-shape, the charged particle beam generation chamber 14 may also have an L-shape. In addition, for example, the charged particle beam scanning unit 13 controls the irradiation position of the charged particle beam L with respect to the target 8, and the current monitor 16 measures the current of the charged particle beam L with which the target 8 is irradiated.

The acceleration unit 11 accelerates charged particles such as protons and generates the charged particle beam L such as a proton beam. In the present embodiment, a cyclotron is employed as the acceleration unit 11. As the acceleration unit 11, a different acceleration unit such as a synchrotron, a synchro-cyclotron, or a linear accelerator may be used instead of the cyclotron.

One end (end portion on the upstream side) of the beam transportation path 12 is connected to the acceleration unit 11. The beam transportation path 12 includes a beam adjustment unit 15 that adjusts the charged particle beam L. The beam adjustment unit 15 has a horizontal steering electromagnet and a horizontal-vertical steering electromagnet for adjusting the axis of the charged particle beam L, and a quadrupole electromagnet for restraining radiation of the charged particle beam L, a four-way slit for shaping the charged particle beam L, and the like. The beam transportation path 12 need only have a function of transporting the charged particle beam L, and the beam adjustment unit 15 is not essential.

The irradiation position of the charged particle beam L transported through the beam transportation path 12 is controlled by the charged particle beam scanning unit 13, and the target 8 is irradiated with the charged particle beam L. The charged particle beam scanning unit 13 may be omitted, such that the same place in the target 8 is irradiated with the charged particle beam L at all times.

The target 8 generates the neutron beam N in response to irradiation of the charged particle beam L. For example, the target 8 is formed of beryllium (Be), lithium (Li), tantalum (Ta), or tungsten (W) in a plate shape (for more details, the material of the target 8 will be described below). The neutron beam irradiation unit 20 irradiates the patient S inside the irradiation chamber 2 with the neutron beam N generated by the target 8.

The neutron beam irradiation unit 20 includes a moderator 21 that decelerates the neutron beam N which has exited from the target 8, and a beam blockage body 22 that blocks the neutron beam N and radial rays such as gamma rays from being radiated to the outside. The moderator 21 and the beam blockage body 22 constitute a moderator.

For example, the moderator 21 has a laminated structure formed of a plurality of different materials, and the material of the moderator 21 can be suitably selected depending on various conditions, such as energy, of the charged particle beam L. Specifically, for example, in a case where the acceleration unit 11 outputs a proton beam of 30 MeV and a beryllium target is used as the target 8, lead, iron, aluminum, or calcium fluoride can be adopted as the material of the moderator 21.

The beam blockage body 22 is provided to surround the moderator 21 and has a function of blocking the neutron beam N and radial rays such as gamma rays caused in response to the generated neutron beam N from being radiated to the outside of the beam blockage body 22. At least a part of the beam blockage body 22 may be embedded in a wall W1 separating the charged particle beam generation chamber 14 and the irradiation chamber 2 from each other, or the beam blockage body 22 does not have to be embedded therein. In addition, a wall body 23 forming a part of a side wall surface of the irradiation chamber 2 is provided between the irradiation chamber 2 and the beam blockage body 22. A collimator attachment portion 23a serving as an output port for the neutron beam N is provided in the wall body 23. A collimator 31 for regulating the irradiation field of the neutron beam N is fixed to this collimator attachment portion 23a. The collimator 31 may be attached to the therapy table 3 (which will be described below), without providing the collimator attachment portion 23a in the wall body 23.

In the neutron beam irradiation unit 20 described above, the target 8 is irradiated with the charged particle beam L, and the target 8 generates the neutron beam N in response thereto. The neutron beam N generated by the target 8 is decelerated while passing through the inside of the moderator 21, and the neutron beam N, which has exited from the moderator 21, passes through the collimator 31, so that the patient S on the therapy table 3 is irradiated with the neutron beam N. Here, a thermal neutron beam or an epithermal neutron beam having comparatively low energy can be used as the neutron beam N.

The therapy table 3 functions as a placement table used in neutron capture therapy and can be moved from the preparation room (not illustrated) to the irradiation chamber 2 while having the patient S placed thereon. The therapy table 3 includes a base unit 32 which constitutes a base of the therapy table 3, casters 33 which enables the base unit 32 to move on a floor surface, a top plate 34 on which the patient S is placed, and a driver 35 which causes the top plate 34 to relatively move with respect to the base unit 32. The base unit 32 may be fixed to the floor without using the casters 33.

The neutron capture therapy apparatus 1 includes a control unit 40 that performs various kinds of control processing. The control unit 40 of the neutron capture therapy apparatus 1 is constituted of a CPU, a ROM, and a RAM, for example. The control unit 40 is electrically connected to the acceleration unit 11, the beam adjustment unit 15, the charged particle beam scanning unit 13, and the current monitor 16. In addition, the control unit 40 is electrically connected a therapy planning system 100 according to the present embodiment. The therapy planning system 100 performs therapy planning of neutron capture therapy in which the patient S is irradiated with a neutron beam. The therapy planning system 100 outputs data related to therapy planning to the control unit 40. Consequently, the control unit 40 controls the acceleration unit 11, the beam adjustment unit 15, and the charged particle beam scanning unit 13 based on the therapy planning output from the therapy planning system 100, and a detection result output from the current monitor 16.

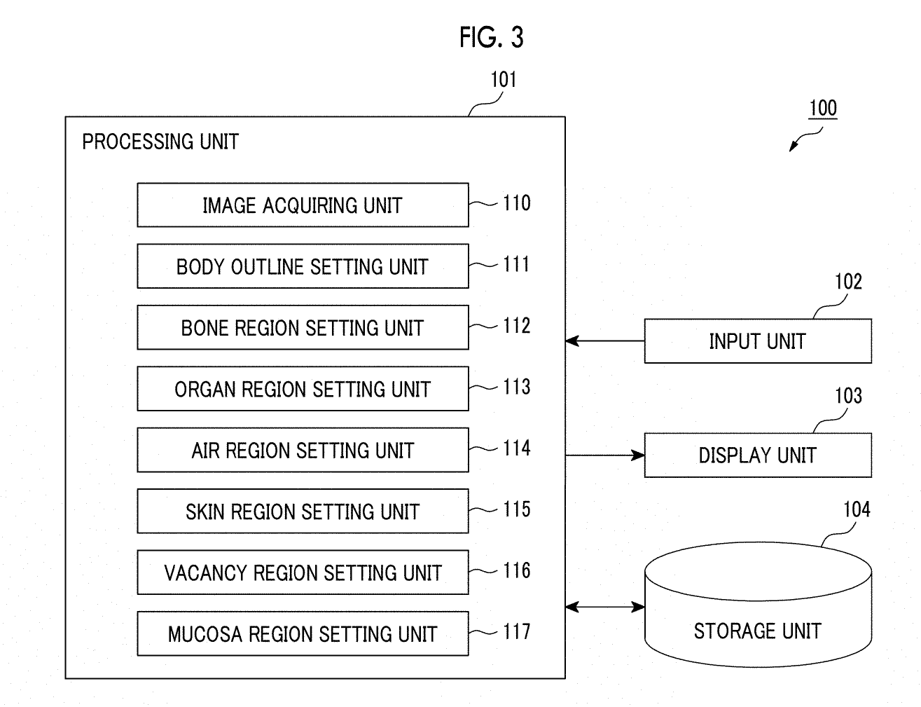

Subsequently, with reference to FIG. 3, a configuration of the therapy planning system 100 will be described in detail. FIG. 3 is a block configuration diagram illustrating the block configuration of the therapy planning system 100. The therapy planning system 100 includes a processing unit 101 that performs various kinds of information processing, an input unit 102 that is used by an operator to input various kinds of information, a display unit 103 that displays various kinds of information for the operator, and a storage unit 104 that transmits and receives various kinds of information with respect to the processing unit 101. The input unit 102 is constituted of various kinds of interfaces, such as a keyboard and a mouse. The display unit 103 is constituted of a monitor or the like. The storage unit 104 may store a CT image and may store data and the like of therapy planning computed by the processing unit 101.

The processing unit 101 has a function of setting various kinds of regions based on an image of the patient S and computing a radiation dose distribution and the like of a neutron beam with respect to the patient S based on the set region. Here, therapy of neutron capture therapy is a therapeutic method in which boron that has been injected into cancer cells in advance is irradiated with a neutron beam so that the cancer cells are selectively destroyed due to dispersion of heavy charged particles generated through nuclear reaction between the neutron beam and the boron. In this manner, since the therapy is a therapeutic method accompanying nuclear reaction, in order to grasp the radiation dose distribution in a scanned image of the patient S, the atomic composition of each region in the image of the patient S needs to be grasped. Therefore, the processing unit 101 sets regions in an image based on the atomic composition. Specifically, in an image, the processing unit 101 sets a bone region in which a bone is disposed, an organ region in which an organ is disposed, an air region which is occupied by air, a skin region in which skin is disposed, a vacancy region in which a vacancy is disposed, and a mucosa region in which mucosa is disposed.

Specifically, the processing unit 101 includes an image acquiring unit 110, a body outline setting unit 111, a bone region setting unit 112, an organ region setting unit 113, an air region setting unit 114, a skin region setting unit 115, a vacancy region setting unit 116, and a mucosa region setting unit 117.

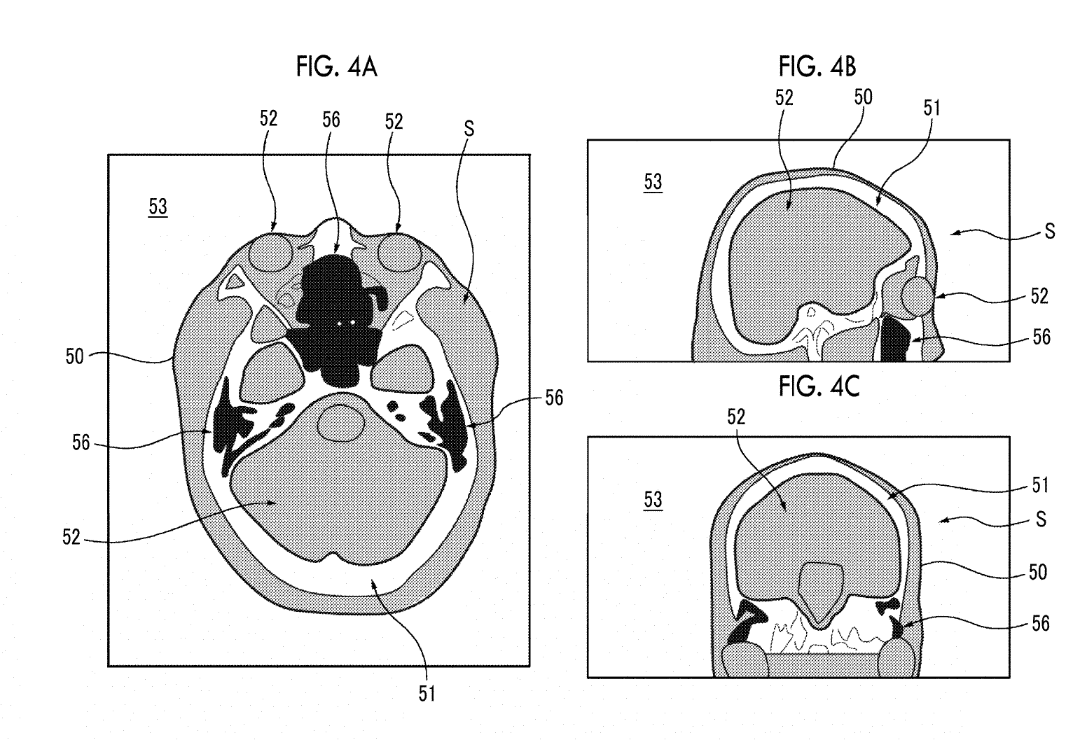

The image acquiring unit 110 acquires an image of the patient S. The image acquiring unit 110 acquires the image by reading out the image stored in the storage unit 104. However, the image acquiring unit 110 may directly take an image from an external instrument. A CT image or the like is employed as an image to be acquired. FIGS. 4A to 4C illustrate examples of images of the patient S. FIG. 4A is an image illustrating a state of the head of the patient S cut in round slices when seen from above. FIG. 4B is an image illustrating a state of the head of the patient S cut in round slices when seen in a lateral direction. FIG. 4C is an image illustrating a state of the head of the patient S cut in round slices when seen from the front. As images of the patient S, there are a plurality of copies of images in each of which the patient S is cut in round slices at a predetermined pitch.

The body outline setting unit 111 sets a body outline 50 of the patient S based on an image acquired by the image acquiring unit 110. The body outline 50 indicates an outline of the outermost part of the body of the patient S. The body outline setting unit 111 can be set the body outline 50 in an image by a known method. For example, the body outline setting unit 111 sets the body outline 50 by an outline extraction including first differentiation, second differentiation, template matching, and the like. After the body outline setting unit 111 sets the body outline 50, the bone region setting unit 112, the organ region setting unit 113, and the vacancy region setting unit 116 (which will be described below) may perform processing for setting each region in only the region on the inner side of the body outline 50.

The bone region setting unit 112 sets a part in which a bone is disposed on the inner side of the body outline 50, as a bone region 51. For example, a part, at which the scull, teeth, or the like is disposed, is set as the bone region 51. The bone region setting unit 112 can set the bone region 51 in an image by a known method. For example, the bone region setting unit 112 sets the bone region 51 by performing threshold processing with respect to a gray scale (CT value). Examples of the atomic composition of the bone region 51 include H, C, N, O, P, Ca, Na, Mg, and S.

The organ region setting unit 113 sets a part in which an organ is disposed on the inner side of the body outline 50, as an organ region 52. For example, a part, at which the brain, eyes, or the like is disposed, is set as the organ region 52. The organ region setting unit 113 can set the organ region 52 in an image by a known method. For example, the organ region setting unit 113 sets the organ region 52 by a so-called model based segmentation method or a so-called smart segmentation (knowledge-based segmentation) method. Examples of the atomic composition of the organ region 52 include H, C, N, O, Na, P, S, Cl, and K.

The air region setting unit 114 sets a region occupied by air around the patient S, as an air region 53. The air region setting unit 114 sets the entire region on the outer side of the body outline 50, as the air region 53. In an image, there are cases where the therapy table 3, an instrument, or the like is also scanned in a region on the outer side of the body outline 50. Even in such a case, the air region setting unit 114 regards the part of the therapy table 3, an instrument, or the like as the air region 53, thereby reducing a load of processing.

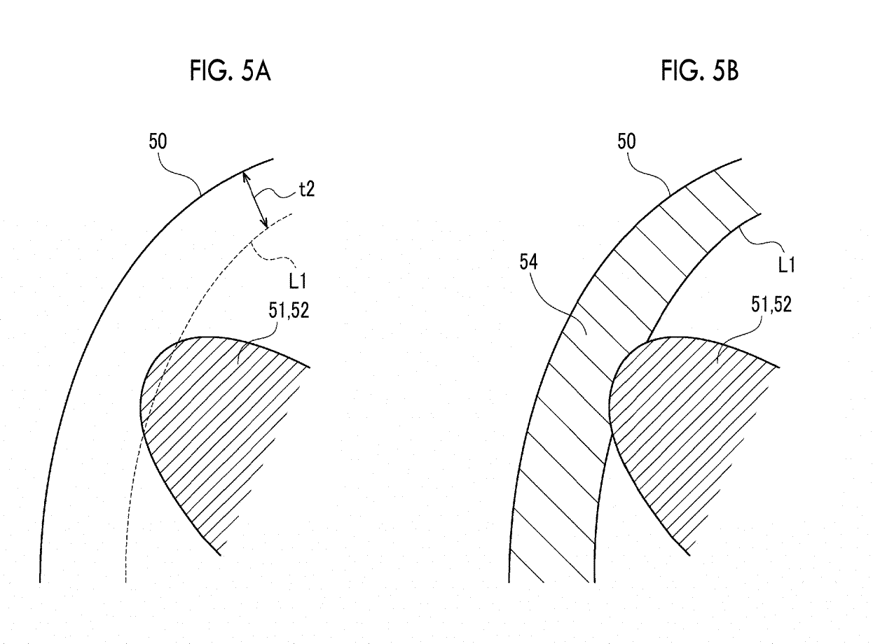

As illustrated in FIGS. 5A and 5B, the skin region setting unit 115 sets a part on the inner side of the body outline 50 by a second thickness t2 from the body outline 50, as a skin region 54 at which skin is disposed. In addition, the skin region setting unit 115 deletes a part of the skin region 54 overlapping at least one of the bone region 51 and the organ region 52 from the skin region 54. For example, as illustrated in FIG. 5A, the skin region setting unit 115 sets a boundary line L1 on the inner side by the second thickness t2 from the body outline 50. As illustrated in FIG. 5B, the skin region 54 sets a region between the body outline 50 and the boundary line L1 as the skin region 54. In this case, a part of the bone region 51 or the organ region 52 extending to the outer side beyond the boundary line L1 is set as the bone region 51 or the organ region 52. Examples of the atomic composition of the skin region 54 include H, C, N, O, Na, P, S, Cl, and K.

The vacancy region setting unit 116 sets apart in which a value of a pixel of an image is within a predetermined range on the inner side of the body outline 50 set by the body outline setting unit 111, as a vacancy region 56 in which a vacancy is disposed (refer to FIGS. 4A to 4C). For example, the threshold (CT value) of the pixel may be set to approximately −1,000. In addition, for example, an internal space of the nose, ears, the mouth, or the like is set as the vacancy region 56. The vacancy region 56 is set as a region constituted of the atomic composition of air including N₂, O₂, CO₂, or the like.

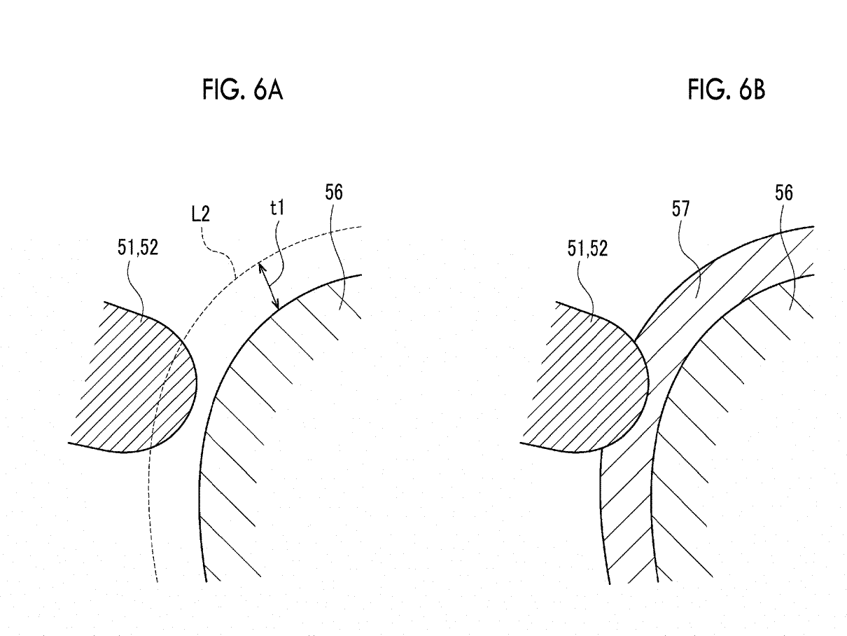

As illustrated in FIGS. 6A and 6B, the mucosa region setting unit 117 sets a part on the outer side of the vacancy region 56 by a first thickness t1 from the vacancy region 56, as a mucosa region 57 in which mucosa is disposed. In addition, the mucosa region setting unit 117 deletes a part of the mucosa region 57 overlapping at least one of the bone region 51 and the organ region 52 from the mucosa region 57. For example, as illustrated in FIG. 6A, the mucosa region setting unit 117 sets a boundary line L2 on the outer side by the first thickness t1 from an outer edge of the vacancy region 56. As illustrated in FIG. 6B, the mucosa region 57 sets a region between the vacancy region 56 and the boundary line L2 as the mucosa region 57. In this case, a part of the bone region 51 or the organ region 52 extending to the inner side beyond the boundary line L2 is set as the bone region 51 or the organ region 52.

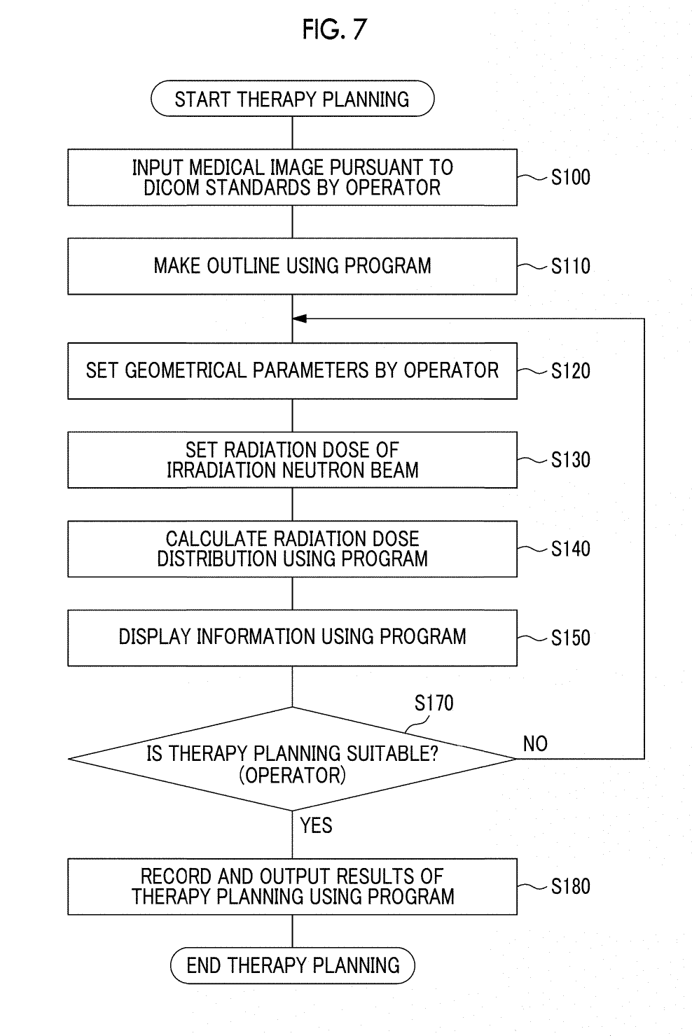

Subsequently, with reference to FIG. 7, a procedure of the therapy planning using the therapy planning system 100 will be described. In the following description, the processing performed by an operator indicates processing in which the therapy planning system 100 causes the display unit 103 to display a request for an input of information such that an input of the operator is received through the input unit 102 based on the display.

As illustrated in FIG. 7, an operator inputs an image to the therapy planning system 100 (Step S100). In S100, a medical image pursuant to the DICOM standards is input. Subsequently, each part of an irradiation target is outlined using a program of the processing unit 101 of the therapy planning system 100 (Step S110). In S110, the body outline 50, the vacancy region 56, the mucosa region 57, the skin region 54, the bone region 51 and the organ region 52 are set as described above.

Subsequently, geometrical parameters are set to each of the outlines set by an operator in S110 (Step S120). In S120, the atomic composition of each region, tolerance to a neutron beam, and the like may be set. In addition, the radiation dose of an irradiation neutron beam is set by an operator or using the program of the processing unit 101 of the therapy planning system 100 (Step S130). In S130, the radiation dose of a neutron beam with which the neutron capture therapy apparatus 1 irradiates a patient is set. In addition, the radiation dose distribution of a neutron beam is calculated using the program of the processing unit 101 (Step S140). In S140, the radiation dose distribution in a case where a patient is irradiated with a neutron beam is calculated based on the items set before S130.

Subsequently, various kinds of information are displayed using the program of the processing unit 101 (Step S150). Here, the radiation dose distribution considering nuclear reaction to a neutron beam may be displayed based on the atomic composition of each part, or analysis and analysis results of the radiation dose distribution may be displayed.

Subsequently, an operator determines suitability of therapy planning (Step S170). If the operator determines that the therapy planning is not suitable, the process returns to the processing of S120 again. On the other hand, if the operator determines that the therapy planning is suitable, results of the therapy planning is recorded and output with respect to the control unit 40 using the program of the processing unit 101 (Step S180). After the steps described above, therapy planning is completed.

Subsequently, operations and effects of the therapy planning system 100 according to the present embodiment will be described.

Here, since neutron capture therapy is a therapeutic method utilizing nuclear reaction between a neutron beam and atomic nuclei of an irradiation object, the radiation dose distribution needs to be calculated using a model considering a region based on the atomic composition of an irradiation target. In addition, since a part of an irradiation target corresponding to mucosa is highly sensitive to radial rays, when therapy planning is performed, the therapy planning needs to be performed after setting a region in which mucosa is disposed. However, in a case where an operator sets the mucosa region while visually recognizing an image, there is a problem of an increase in work of the operator.

Therefore, the therapy planning system 100 according to the present embodiment includes the vacancy region setting unit 116 that sets a part in which a value of a pixel of an image is within a predetermined range on the inner side of the body outline 50 set by the body outline setting unit 111, as the vacancy region 56 in which a vacancy is disposed. In an image acquired by the image acquiring unit 110, the part in which the value of the pixel is within the predetermined range is a part on the outer side of the body outline 50 of the irradiation target, or a part in which a vacancy is disposed inside the irradiation target. Therefore, the vacancy region 56 can be set by the body outline setting unit 111 that sets the body outline 50 and the vacancy region setting unit 116 that determines the value of the pixel on the inner side of the body outline 50. Here, mucosa is disposed near the surface of an inner wall part of the vacancy. Thus, the mucosa region setting unit 117 can set a part on the outer side of the vacancy region 56 by the first thickness t1 from the surface of the vacancy region 56 set by the vacancy region setting unit 116, as the mucosa region 57 in which mucosa is disposed (refer to FIGS. 6A and 6B). In this manner, the mucosa region 57 can be easily set by setting the vacancy region 56. Therapy planning can be performed based on the radiation dose of a neutron beam with respect to the mucosa region 57. In addition, setting can be performed on the system side, instead of determination of an operator performed by visually recognizing a plurality of copies of images of the mucosa region 57. Therefore, the operator need only perform work of a final check for the mucosa region 57. Consequently, therapy planning suitable for neutron capture therapy can be performed.

The therapy planning system 100 according to the present embodiment further includes the bone region setting unit 112 that sets a part in which a bone is disposed on the inner side of the body outline 50, as the bone region 51, and the organ region setting unit 113 that sets a part in which an organ is disposed on the inner side of the body outline 50, as the organ region 52. The mucosa region setting unit 117 deletes a part of the set mucosa region 57 overlapping at least one of the bone region 51 and the organ region 52 from the mucosa region 57. Since bones and organs are formed of an atomic composition different from that of mucosa, the mucosa region 57 is set as a region different from the bone region 51 or the organ region 52. Therefore, the mucosa region 57 can be more accurately set by deleting a part overlapping at least one of the bone region 51 and the organ region 52 from the mucosa region 57.

The therapy planning system 100 according to the present embodiment further includes the skin region setting unit 115 that sets a part on the inner side of the body outline 50 by the second thickness t2 from the body outline 50, as the skin region 54 in which skin is disposed. Not only mucosa but also skin is a part in which the allowable radiation dose of an irradiation neutron beam is restricted. In addition, skin is disposed at a part on the inner side of the body outline 50 by a predetermined thickness from the body outline 50. In this manner, the skin region 54 can be easily set by setting the body outline 50. Therapy planning can be performed based on the radiation dose of a neutron beam with respect to the skin region 54. Therefore, more suitable therapy planning can be performed by setting the skin region 54.

The therapy planning system 100 according to the present embodiment further includes the air region setting unit 114 that sets the entire region on the outer side of the body outline 50, as the air region 53. Accordingly, even in a case where the therapy table 3 or the like is disposed, a load of computation can be reduced by the air region setting unit 114 that sets the entire region on the outer side of the body outline 50 as the air region 53.

The present invention is not limited to the embodiment described above. For example, in the embodiment described above, the bone region 51, the organ region 52, and the skin region 54 have been set in addition to the mucosa region 57. However, at least the mucosa region 57 need only be set.

In addition, in the therapy planning described above, details and procedures of processing other than setting of the body outline 50 and the mucosa region 57 are not particularly limited and may be suitably changed.

It should be understood that the invention is not limited to the above-described embodiment, but may be modified into various forms on the basis of the spirit of the invention. Additionally, the modifications are included in the scope of the invention.

What is claimed is:

1. A therapy planning system for neutron capture therapy performing therapy planning of the neutron capture therapy in which an irradiation target is irradiated with a neutron beam, the therapy planning system comprising:
an image acquiring unit configured to acquire an image of the irradiation target;
a body outline setting unit configured to set a body outline of the irradiation target based on the image acquired by the image acquiring unit;
a vacancy region setting unit configured to set a part in which a value of a pixel of the image is within a predetermined range on an inner side of the body outline set by the body outline setting unit, as a vacancy region in which a vacancy is disposed; and
a mucosa region setting unit configured to set a part on an outer side of the vacancy region by a first thickness from a surface of the vacancy region set by the vacancy region setting unit on the inner side of the body outline, as a mucosa region in which mucosa is disposed.

2. The therapy planning system for neutron capture therapy according to claim 1, further comprising:
a bone region setting unit configured to set a part in which a bone is disposed on the inner side of the body outline, as a bone region; and
an organ region setting unit configured to set a part in which an organ is disposed on the inner side of the body outline, as an organ region,
wherein the mucosa region setting unit deletes a part of the set mucosa region overlapping at least one of the bone region and the organ region from the mucosa region.

3. The therapy planning system for neutron capture therapy according to claim 1, further comprising:
a skin region setting unit configured to set a part on an inner side of the body outline by a second thickness from the body outline, as a skin region in which skin is disposed.

4. The therapy planning system for neutron capture therapy according to claim 1, further comprising:
an air region setting unit configured to set an entire region on the outer side of the body outline, as an air region.
